(19) United States
(12) Patent Application Publication
(10) Pub. No.: US 2025/0346969 A1
(43) Pub. Date: Nov. 13, 2025

(54) Giant Panda Canine Distemper Virus Detection Kit Based on Multi-Enzyme Isothermal Nucleic Acid Rapid Amplification Technology and use Method

(71) Applicants: China Conservation and Research Center for Giant Panda, Chengdu City (CN); Sichuan University, Chengdu City (CN)

(72) Inventors: Caiwu Li, Chengdu City (CN); Xin Yang, Chengdu City (CN); Mingyue Tian, Chengdu City (CN); Zhengquan Hu, Chengdu City (CN); Desheng Li, Chengdu City (CN); Yan Huang, Chengdu City (CN); Chengdong Wang, Chengdu City (CN); Shanshan Ling, Chengdu City (CN); Chengyao Hou, Chengdu City (CN); Lijun Zhao, Chengdu City (CN); Xuelin Long, Chengdu City (CN)

(21) Appl. No.: 19/059,022

(22) Filed: Feb. 20, 2025

(30) Foreign Application Priority Data
May 10, 2024 (CN) 202410576827.0

(51) Int. Cl. C12Q 1/70

(52) U.S. Cl. CPC C12Q 1/701; C12Q 2600/158

(57) ABSTRACT

A giant panda canine distemper virus detection kit based on multi-enzyme isothermal nucleic acid rapid amplification technology and a use method are provided. For CDV, the kit selects virus conserved gene, designs specific primers in the conserved region of the gene, amplifies the N gene fragment of CDV by multi-enzyme isothermal rapid amplification technology, and then detects the amplified products with nucleic acid colloidal gold test strips to establish a MIRA detection method for rapid auxiliary diagnosis with CD.

### CROSS-REFERENCE TO RELATED APPLICATIONS

This application claims priority to Chinese Patent Application No. 202410576827.0, filed on May 10, 2024, the contents of which are hereby incorporated by reference.

### REFERENCE TO ELECTRONIC SEQUENCE LISTING

The present application is being filed along with an Electronic Sequence Listing in electronic format. The Electronic Sequence Listing is provided as a file named ReplacementSequence which is 4,509 bytes in size, created and last modified on Apr. 25, 2025. The information in the Electronic Sequence Listing is incorporated herein by reference in its entirety.

### TECHNICAL FIELD

The present disclosure belongs to the technical field of biological detection, and particularly relates to a giant panda canine distemper virus detection kit based on multi-enzyme isothermal nucleic acid rapid amplification technology and a use method.

### BACKGROUND

Giant panda (Ailuropoda melanoleuca) belongs to Carnivora, a mammal of the Giant Panda Family, and is a rare and endangered wild species endemic to China. Canine distemper (CD) caused by canine distemper virus (CDV) infection is the top infectious disease threatening the life safety of giant pandas. Canine distemper is a highly contagious disease, which has a late course, leads to serious illness and is difficult to cure, causing great damage to sick animals. Early disease diagnosis and screening are important steps to prevent and treat canine distemper virus infection.

### SUMMARY

In order to realize the early diagnosis of this kind of viral infectious diseases, prevent the spread of the virus, and reduce losses, one objective of the present disclosure is to provide a primer composition for giant panda canine distemper virus detection based on multi-enzyme isothermal nucleic acid rapid amplification technology. This primer composition is aimed at CDV, selecting a virus conservative gene, designing specific primers in the conserved region of the gene, amplifying the N gene fragment of CDV through the multi-enzyme isothermal rapid amplification (MIRA) technology, and then detecting the amplified products with nucleic acid colloidal gold test strips to establish a MIRA detection method for rapid auxiliary diagnosis of CD.

MIRA technology has good applicability for pathogen nucleic acid detection: under the action of special enzymes, the target gene fragment may be amplified at room temperature and constant temperature, greatly reducing its dependence on professional equipment. MIRA technology needs short reaction duration, and the reaction starts as soon as the reagent is added to the system containing samples, and the amplification reaction is completed within 15 minutes (min), and the detection results may be obtained within 40 min. MIRA technology may be used to detect ribonucleic acid (RNA) or deoxyribonucleic acid (DNA) samples with a wide detection range, and RNA templates may be directly amplified by adding specific high-efficiency reverse transcriptase into the system.

Another objective of the present disclosure is to provide a canine distemper virus nucleic acid detection system with multi-enzyme isothermal nucleic acid rapid amplification technology and colloidal gold nucleic acid test strip detection technology, which has the advantages of simple operation, simple conditions, rapid reaction, high sensitivity, strong specificity, good repeatability and low dependence on complex detection environment, and provides a new technical means for detecting canine distemper virus, one of the important giant panda viruses. Moreover, on the basis of this study, specific MIRA primers are designed for the conserved sequences of conserved genes of various pathogens, which may develop technical methods suitable for detection of different pathogens. The establishment of this detection system provides a new choice for pathogen detection in animal disease prevention.

The present disclosure is realized by the following technical schemes.

A primer composition for giant panda canine distemper virus detection based on multi-enzyme isothermal nucleic acid rapid amplification technology includes a primer pair and a probe for multi-enzyme isothermal nucleic acid rapid amplification, where the primer pair consists of a forward primer and a reverse primer, and the nucleotide sequence of the forward primer is shown in SEQ ID NO. 3: CTCTGGAGTTATGCTATGGGAGTTGGTGTT;
the nucleotide sequence of the reverse primer is shown in SEQ ID NO. 4:
GGTTGTTGATTAGTGACTTCGGATCTTTCTG;
a sequence of the probe is directly labeled with fluorescein amidite (FAM) at a 5′ end of the forward primer and biotin at a 5′ end of the reverse primer;
the primer pair is designed by taking the giant panda/SX/2014 strain of giant panda derived-canine distemper virus with Genbank accession number KP793921 as a template.

An application of the primer composition for giant panda canine distemper virus detection based on multi-enzyme isothermal nucleic acid rapid amplification technology in preparation of a giant panda canine distemper virus detection product.

Optionally, the detection product includes any one of a detection reagent a detection kit and a detection chip.

A giant panda canine distemper virus detection kit based on multi-enzyme isothermal nucleic acid rapid amplification technology includes the primer composition.

Optionally, the kit includes a multi-enzyme isothermal nucleic acid rapid amplification reaction system, totaling 50 microliter (μL), specifically including:
a forward primer with a concentration of 10 micromolar (μM) and a volume of 2 μL;
a reverse primer with a concentration of 10 μM and a volume of 2 μL;
a probe with a concentration of 10 μM and a volume of 0.6 μL;
a buffer A with a volume of 29.4 μL;
a buffer B with a volume of 2.5 μL; and
a nucleic acid template and nuclease-free water, with a volume of 13.5 μL.

Optionally, the kit further includes an assorted nucleic acid detection test strip for detecting multi-enzyme isothermal nucleic acid rapid amplification products.

Optionally, the nucleic acid detection test strip is a lateral flow chromatography test strip.

Optionally, the lateral flow chromatography test strip is a colloidal gold lateral flow chromatography test strip.

A use method of the giant panda canine distemper virus detection kit based on multi-enzyme isothermal nucleic acid rapid amplification technology includes following steps: S1, designing multi-enzyme isothermal nucleic acid rapid amplification specific primers according to the nucleic acid sequence of the N nucleocapsid protein gene of the giant panda canine distemper virus published on National Center for Biotechnology Information (NCBI) GenBank;
S2, extracting RNA of canine distemper virus from the giant panda canine distemper isolates; after reverse transcription, amplification, plasmid insertion and competent cell culture, extracting recombinant plasmid as standard plasmid; and
S3, using prepared standard plasmid gradient samples as templates, using a DNA isothermal rapid amplification kit-colloidal gold test strip type, adding each reaction reagent in turn, and performing a multi-enzyme isothermal nucleic acid rapid amplification reaction.

Optionally, the temperature of the multi-enzyme isothermal nucleic acid rapid amplification reaction is 37 degrees Celsius (° C.) for 15 min; the minimum detection limit of the kit for plasmid template is 1.15×10² copies.

Compared with the prior art, the present disclosure has at least the following technical effects.

In order to realize the early diagnosis of this kind of viral infectious diseases, prevent the spread of the virus, and reduce losses, the present disclosure provides a primer composition for giant panda canine distemper virus detection based on multi-enzyme isothermal nucleic acid rapid amplification technology. This primer composition is aimed at CDV, selecting a virus conservative gene, designing specific primers in the conserved region of the gene, amplifying the N gene fragment of CDV through the multi-enzyme isothermal rapid amplification technology, and then detecting the amplified products with nucleic acid colloidal gold test strips to establish a MIRA detection method for rapid auxiliary diagnosis of CD.

MIRA technology has good applicability for pathogen nucleic acid detection: under the action of special enzymes, the target gene fragment may be amplified at room temperature and constant temperature, greatly reducing its dependence on professional equipment. MIRA technology needs short reaction duration, and the reaction starts as soon as the reagent is added to the system containing samples, and the amplification reaction is completed within 15 min, and the detection results may be obtained within 40 min. MIRA technology may be used to detect RNA or DNA samples with a wide detection range, and RNA templates may be directly amplified by adding specific high-efficiency reverse transcriptase into the system.

The present disclosure provides a canine distemper virus nucleic acid detection system with multi-enzyme isothermal nucleic acid rapid amplification technology and colloidal gold nucleic acid test strip detection technology, which has the advantages of simple operation, simple conditions, rapid reaction, high sensitivity, strong specificity, good repeatability and low dependence on complex detection environment, and provides a new technical means for detecting canine distemper virus, one of the important giant panda viruses. Moreover, on the basis of this study, specific MIRA primers are designed for the conserved sequences of conserved genes of various pathogens, which may develop technical methods suitable for detection of different pathogens. The establishment of this detection system provides a new choice for pathogen detection in animal disease prevention.

### BRIEF DESCRIPTION OF THE DRAWINGS

FIG. 1 is a schematic diagram of electrophoretic separation results of ligation products prepared for standard plasmid.

FIG. 2 is a schematic diagram of MIRA reaction system establishment and condition optimization.

FIG. 3 is a schematic diagram of MIRA sensitivity test.

FIG. 4 is a schematic diagram of quantitative polymerase chain reaction (qPCR) amplification curves.

FIG. 5 shows a repeatability test of MIRA reaction system.

FIG. 6 shows a specificity test of MIRA reaction system.

FIG. 7 is a flow chart of a use method of a giant panda canine distemper virus detection kit based on multi-enzyme isothermal nucleic acid rapid amplification technology according to the present disclosure.

### DETAILED DESCRIPTION OF THE EMBODIMENTS

The implementation schemes of the present disclosure will be described in detail with embodiments, but those skilled in the art will understand that the following embodiments are only used to illustrate the present disclosure, and should not be regarded as limiting the scope of the present disclosure. The specific conditions not indicated in the embodiments are carried out according to the conventional conditions or the conditions suggested by the manufacturers, and the reagents or instruments used are all conventional products that may be obtained by commercial purchase.

### Embodiment

### I. Primer and Probe Design

1. According to the nucleic acid sequence of CDV nucleocapsid protein (N) gene (KP793921) published on NCBI GenBank, a pair of conventional specific polymerase chain reaction (PCR) primers were designed by using software Primer5 according to the design principle of PCR primers; a pair of specific qPCR primers were designed according to the design principle and sequence requirements of qPCR primers; according to the design principle of MIRA primer (or probe), a pair of MIRA primers with high sensitivity and specificity were designed and screened.

| | 5′-3′ | SEQ ID NO. |
|---|---|---|
| PCR-F1 | TTCAGGCTCGGGCAAGAAATGGTGAGAAGAT | 1 |
| PCR-R1 | AGTGCGAATCGTCCGGTCCTCCGTTGTCTTG | 2 |
| MIRA-F3 | CTCTGGAGTTATGCTATGGGAGTTGGTGTT | 3 |
| MIRA-R3 | GGTTGTTGATTAGTGACTTCGGATCTTTCTG | 4 |
| Probe-F2 | FAM-CTCTGGAGTTATGCTATGGGAGTTGGTGTT | — |
| Probe-R2 | Biotin-GGTTGTTGATTAGTGACTTCGGATCTTTCTG | — |

### 2. Preparation of Standard Plasmid

(1) RNA of canine distemper virus was extracted from giant panda canine distemper isolates (giant panda/SX/2014);
(2) reverse transcription of the extracted RNA into cDNA;
(3) reverse transcription of the extracted RNA into cDNA;
(4) recovery of PCR products;
(5) the target gene CDV-N was linked with plasmid pMD-19T by enzyme digestion;
(6) the target gene CDV-N was linked with plasmid pMD-19T by enzyme digestion;
(7) expanded culture of Escherichia coli DH5α cells; and
(8) a plasmid extraction kit was used to extract recombinant plasmid (plasmid extraction kit (centrifugal column type) from Tiangen Biochemical Technology Co., Ltd.) from Escherichia coli bacterial solution.

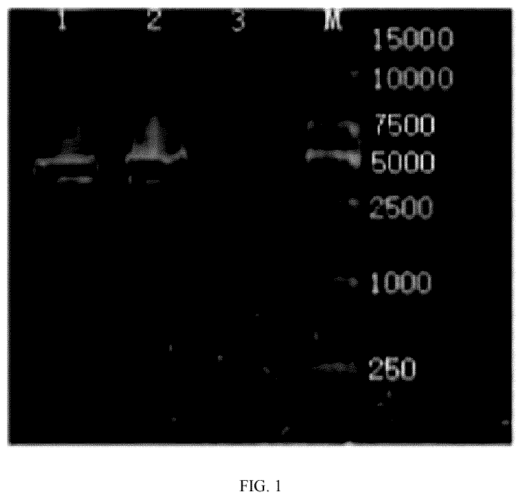

A schematic diagram of electrophoretic separation results of ligation products prepared from standard plasmid is shown in FIG. 1.

The results showed that a fragment of 3405 base pairs (bp) of recombinant plasmid was isolated from the sample, proving that the target gene fragment had been inserted into pMD-19T plasmid correctly.

### 3. Establishment of MIRA Reaction System and Optimization of Conditions

(1) According to the instructions of DNA isothermal rapid amplification kit (colloidal gold test strip type), each MIRA reaction system consists of 29.4 μL A Buffer, 2 μL forward primer (10 μM), 2 μL reverse primer (10 μM), 0.6 μL probe (10 μM), 13.5 μL nucleic acid template, ddH₂O and 2.5 μL B Buffer, totaling 50 μL.

After mixing evenly, the reaction system was incubated in a 42° C. incubator for 8-12 min. The whole reaction system was 10 μL, consisting of 5 μL A Buffer, 0.5 μL upstream primer (10 μM), 0.5 μL downstream primer (10 μM), 2.5 μL ddH₂O, 1 μL nucleic acid template and 0.5 μL B Buffer.

(2) The prepared standard plasmid was diluted by 10⁶ times with ddH₂O, and the copy number of the plasmid was about 1.15×10⁵. The reaction condition optimization experiment was performed by using plasmid with this concentration as a template, and the variables were controlled, and the reaction temperatures were set to 25° C., 30° C., 35° C. and 40° C., and the reaction durations were set to 10, 15 and 20 min respectively.

(3) The colloidal gold test strips were used for detection, and the color development was observed to determine the best reaction conditions.

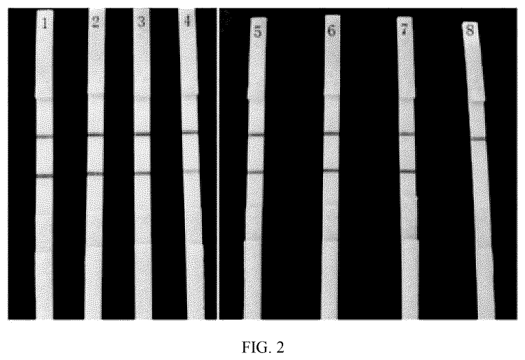

A schematic diagram of MIRA reaction system establishment and condition optimization is shown in FIG. 2, where 1-4: reaction duration was 15 min, reaction temperatures were 40° C., 35° C., 30° C., 25° C.; 5-7: the reaction temperature was 37° C. and the reaction durations were 10, 15 and 20 min; 8: negative control.

The results showed that MIRA could achieve specific amplification within the reaction duration of 10-20 min, and there was no significant difference in the color development of amplification products on colloidal gold test strips, indicating that all groups completed the amplification reaction within 10 min. In order to ensure sufficient amplification reaction and to reduce the impact of false positives on experimental results due to prolonged reaction duration, the optimal reaction duration for MIRA was determined to be 15 min.

### 4. MIRA Sensitivity Test

The prepared standard plasmid was diluted with nuclease-free ddH₂O at a ratio of 10 times: 10 μL of standard plasmid was taken, 90 μL of ddH₂O was added, and the mixture was evenly mixed to obtain a sample with a dilution of 10⁻¹. Then, 10 μL of standard plasmid with a dilution of 10⁻¹ was taken, and 90 μL of ddH₂O was added, and the mixture was evenly mixed to obtain a sample with a dilution of 10⁻². Analogously, 10 standard plasmid gradient samples were prepared.

Determination of the sensitivity of MIRA reaction: using the prepared standard plasmid gradient samples as templates, using the DNA isothermal rapid amplification kit (colloidal gold test strip type), each reaction reagent was added in sequence to carry out MIRA amplification reaction.

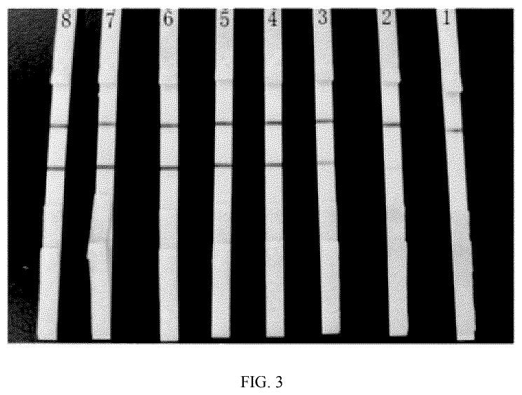

A schematic diagram of MIRA sensitivity test is shown in FIG. 3, where 1: negative control; 2-8: the dilutions of plasmid templates are 10⁻¹⁰, 10⁻⁹, 10⁻⁸, 10⁻⁷, 10⁻⁶, 10⁻⁵ and 10⁻⁴, respectively.

The results showed that the nucleic acid concentration was from low to high, the quality control lines were all colored and the brightness was equivalent, and the brightness of the detection lines increased from low to high, and remained unchanged after reaching the maximum, proving that the brightness of the detection line was positively correlated with the template concentration in a certain range.

### 5. MIRA Repeatability Test

(1) From the prepared standard plasmid gradient samples, the standard samples with plasmid dilutions of 10⁻⁵ and 10⁻⁶ were selected as templates for repeatability test, and each group was set with 3 replicates within the group, while the negative control group was set (using ddH₂O to replace the nucleic acid template in the reaction system).

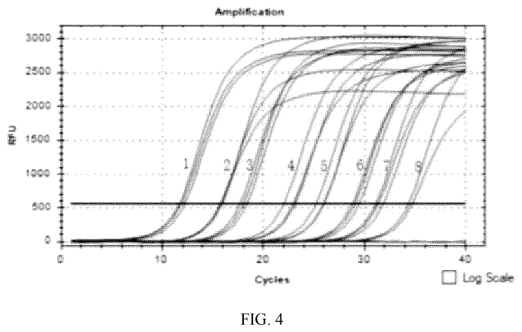

A schematic diagram of qPCR amplification curves is shown in FIG. 4, where the dilutions of standard plasmid corresponding to qPCR amplification curves formed by groups 1-8 are: 10⁻¹, 10⁻², 10⁻³, 10⁻⁴, 10⁻⁵, 10⁻⁶, 10⁻⁷ and 10⁻⁸.

The results showed that the specific amplification curve of CDV-N gene could be obtained from each group of samples, and the standard curve was synthesized: y=−3.259x+41.596 (R2=0.993), and the amplification efficiency was E=102.7%, indicating that the gradient diluted plasmid in this group had a good linear relationship with Cq value, and the repeatability in the group was good, and the results were in line with expectations.

### (2) Determination of Repeatability of MIRA Reaction

Firstly, MIRA amplification reaction was carried out by using DNA isothermal rapid amplification kit (colloidal gold test strip type).

Secondly, in the fume hood, after each reagent was added into the reaction tube in turn, the mixture was immediately mixed evenly and centrifuged briefly, and the reaction solution was shaken to the bottom of the centrifuge tube, and then the centrifuge tube was immediately put into a constant temperature incubator at 37° C. for incubation for 15 min.

Thirdly, after the reaction was completed, 190 μL of ddH₂O was added into the centrifuge tube, and after mixing evenly, 80 μL of diluted reaction product was sucked by a pipette and dripped into the well of colloidal gold test strip, and the strip was left to stand for 15 min, and the experimental results were observed and recorded to evaluate the repeatability of MIRA reaction.

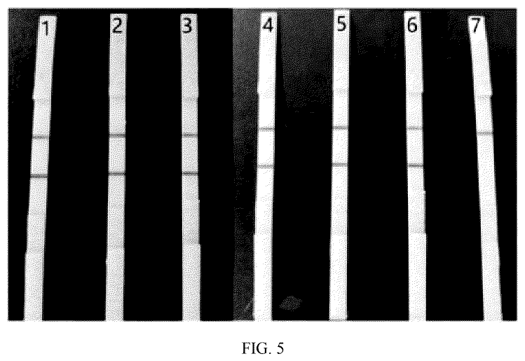

The repeatability of MIRA reaction is shown in FIG. 5, where 1, 2 and 3: the dilution of plasmid templates is 10⁻⁵; 4, 5 and 6: the dilution of plasmid templates is 10⁻⁶; 7: negative control.

The results showed that when the concentration of plasmid templates was the same, the detection results of colloidal gold test strips were the same, all of the results were positive, and the brightness of detection lines was the same in three repeated experiments.

Experiments showed that MIRA reaction had good repeatability under the same conditions.

### 6. Specificity Test

(1) The virus nucleic acids of canine parvovirus (CPV), canine coronavirus (CCoV) and giant panda rotavirus (GPRV) were used as templates (the nucleic acid samples of each virus were obtained from recombinant plasmid containing viral genes stored in the laboratory) to set up the experimental groups, and the standard plasmid prepared in this experiment was used to set up the positive control, and ddH₂O was used to set up the negative control instead of the nucleic acid template.

### (2) Determination of the Specificity of MIRA Reaction

Firstly, MIRA amplification reaction was carried out by using DNA isothermal rapid amplification kit (colloidal gold test strip type).

Secondly, in the fume hood, after each reagent was added into the reaction tube in turn, the mixture was immediately mixed evenly and centrifuged rapidly, and the reaction solution was shaken to the bottom of the centrifuge tube, and then the centrifuge tube was immediately put into a constant temperature incubator at 37° C. for incubation for 15 min.

Thirdly, after the reaction was completed, 190 μL of ddH₂O was added into the centrifuge tube, and after mixing evenly, 80 μL of diluted reaction product was sucked by a pipette and dripped into the well of colloidal gold test strip, and the strip was left to stand for 15 min, and the experimental results were observed and recorded to evaluate the specificity of MIRA reaction. The results were seen within 30 min of the entire process.

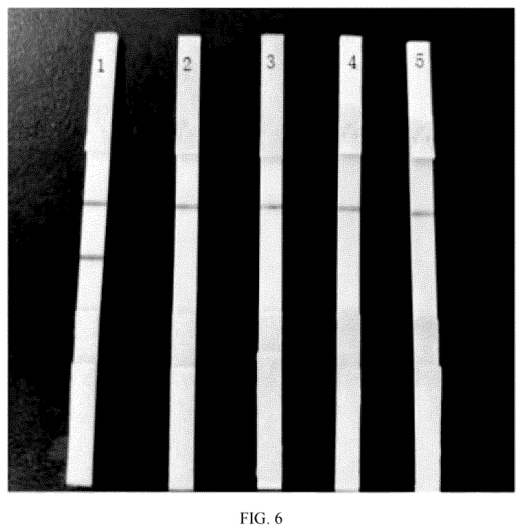

The MIRA specificity test is shown in FIG. 6, where 1: positive control (CDV); 2: CPV; 3: CCOV; 4: GPRV; 5: negative control.

The results showed that the control group with CDV virus nucleic acid as template was tested by colloidal gold test strip after MIRA reaction, and the result was positive. The experimental groups and negative control group with CPV, CCOV and GPRV virus nucleic acids as templates were tested with colloidal gold test strips after MIRA reaction, and the results were all negative.

The experimental results showed that the virus nucleic acid detection system established in this study had strong specificity for CDV.

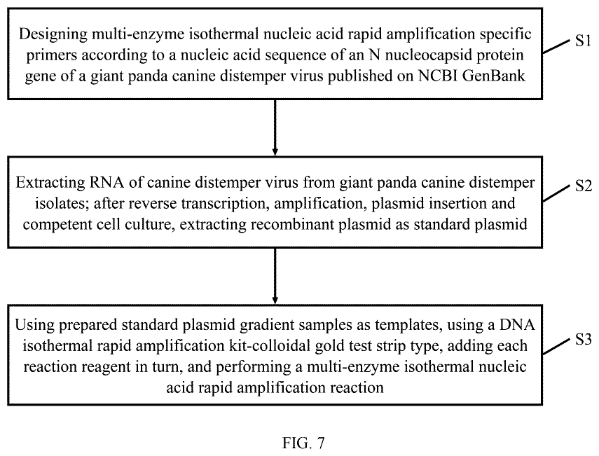

The present disclosure provides a use method of a giant panda canine distemper virus detection kit based on multi-enzyme isothermal nucleic acid rapid amplification technology, including following steps, as shown in FIG. 7:
S1, designing multi-enzyme isothermal nucleic acid rapid amplification specific primers according to a nucleic acid sequence of an N nucleocapsid protein gene of giant panda canine distemper virus published on NCBI GenBank;
S2, extracting RNA of canine distemper virus from giant panda canine distemper isolates; after reverse transcription, amplification, plasmid insertion and competent cell culture, extracting recombinant plasmid as standard plasmid; and
S3, using prepared standard plasmid gradient samples as templates, using a DNA isothermal rapid amplification kit-colloidal gold test strip type, adding each reaction reagent in turn, and performing a multi-enzyme isothermal nucleic acid rapid amplification reaction.

Finally, it should be explained that the above is only the preferred embodiment of the present disclosure, and it is not used to limit the protection scope of the present disclosure. Any modification, equivalent substitution, improvement, etc. made within the spirit and principle of the present disclosure should be included in the protection scope of the present disclosure.

What is claimed is:

1. A primer composition for giant panda canine distemper virus detection based on multi-enzyme isothermal nucleic acid rapid amplification technology, comprising a primer pair and a probe for multi-enzyme isothermal nucleic acid rapid amplification, wherein the primer pair comprises a forward primer and a reverse primer, and a nucleotide sequence of the forward primer is shown in SEQ ID NO. 3:
CTCTGGAGTTATGCTATGGGAGTTGGTGTT;
a nucleotide sequence of the reverse primer is shown in SEQ ID NO. 4:
GGTTGTTGATTAGTGACTTCGGATCTTTCTG;
a sequence of the probe is directly labeled with FAM at a 5′ end of the forward primer and biotin at a 5′ end of the reverse primer; and
the primer pair is designed by taking a giant panda/SX/2014 strain of giant panda derived-canine distemper virus with Genbank accession number KP793921 as a template.

2. A giant panda canine distemper virus detection kit based on multi-enzyme isothermal nucleic acid rapid amplification technology, comprising the primer composition according to claim 1.

3. The giant panda canine distemper virus detection kit based on multi-enzyme isothermal nucleic acid rapid amplification technology according to claim 2, wherein the kit comprises a multi-enzyme isothermal nucleic acid rapid amplification reaction system with a total of 50 μL, specifically comprising:
the forward primer with a concentration of 10 μM and a volume of 2 μL;
the reverse primer with a concentration of 10 μM and a volume of 2 μL;
the probe with a concentration of 10 UM and a volume of 0.6 μL;
a buffer A with a volume of 29.4 μL;
a buffer B with a volume of 2.5 μL; and
a nucleic acid template and nuclease-free water, with a volume of 13.5 μL.

4. The giant panda canine distemper virus detection kit based on multi-enzyme isothermal nucleic acid rapid amplification technology according to claim 3, wherein the kit further comprises an assorted nucleic acid detection test strip for detecting multi-enzyme isothermal nucleic acid rapid amplification products.

5. The giant panda canine distemper virus detection kit based on multi-enzyme isothermal nucleic acid rapid amplification technology according to claim 4, wherein the nucleic acid detection test strip is a lateral flow chromatography test strip.

6. The giant panda canine distemper virus detection kit based on multi-enzyme isothermal nucleic acid rapid amplification technology according to claim 5, wherein the lateral flow chromatography test strip is a colloidal gold lateral flow chromatography test strip.

7. A use method of the giant panda canine distemper virus detection kit based on multi-enzyme isothermal nucleic acid rapid amplification technology according to claim 6, comprising following steps:
S1, designing multi-enzyme isothermal nucleic acid rapid amplification specific primers according to a nucleic acid sequence of an N nucleocapsid protein gene of giant panda canine distemper virus published on NCBI GenBank;
S2, extracting RNA of canine distemper virus from giant panda canine distemper isolates; after reverse transcription, amplification, plasmid insertion and competent cell culture, extracting recombinant plasmid as standard plasmid; and
S3, using prepared standard plasmid gradient samples as templates, using a DNA isothermal rapid amplification kit-colloidal gold test strip type, adding each reaction reagent in turn, and performing a multi-enzyme isothermal nucleic acid rapid amplification reaction.

8. The use method of the giant panda canine distemper virus detection kit based on multi-enzyme isothermal nucleic acid rapid amplification technology according to claim 7, wherein a temperature of the multi-enzyme isothermal nucleic acid rapid amplification reaction is 37° C. for 15 min; and
a minimum detection limit of the kit for a plasmid template is 1.15×10² copies.


SEQUENCE LISTING (4 sequences)

SEQ ID NO: 1 - DNA, 31 bp, Synthetic construct
  source 1..31: mol_type = other DNA
ttcaggctcgggcaagaaatggtgagaagat

SEQ ID NO: 2 - DNA, 31 bp, Synthetic construct
  source 1..31: mol_type = other DNA
agtgcgaatcgtccggtcctccgttgtcttg

SEQ ID NO: 3 - DNA, 30 bp, Synthetic construct
  source 1..30: mol_type = other DNA
ctctggagttatgctatgggagttggtgtt

SEQ ID NO: 4 - DNA, 31 bp, Synthetic construct
  source 1..31: mol_type = other DNA
ggttgttgattagtgacttcggatctttctg